(19) United States
(12) Patent Application Publication
(10) Pub. No.: US 2015/0157628 A1
(43) Pub. Date: Jun. 11, 2015

(54) Pharmaceutical compositions of Lurasidone and Process for preparation thereof

(71) Applicants: Saravanan Kannusamy, Hyderabad (IN); Nagaprasad Vishnubhotla, Hyderabad (IN); Samir Das, Hyderabad (IN); Sivakumaran Meenakshisunderam, Hyderabad (IN)

(72) Inventors: Saravanan Kannusamy, Hyderabad (IN); Nagaprasad Vishnubhotla, Hyderabad (IN); Samir Das, Hyderabad (IN); Sivakumaran Meenakshisunderam, Hyderabad (IN)

(21) Appl. No.: 14/544,224

(22) Filed: Dec. 10, 2014

(30) Foreign Application Priority Data
Dec. 11, 2013 (IN) IN 5702/CHE/2013

(51) Int. Cl. A61K 31/496

(52) U.S. Cl. CPC A61K 31/496

(57) ABSTRACT

Pharmaceutical compositions comprising an atypical antipsychotic as an active agent, process of preparation thereof and method of using the same are provided. Particularly the present invention relates to pharmaceutical compositions comprising lurasidone, process of preparation thereof and method to treat various psychotic disorders such as schizophrenia, positive and negative symptoms of schizophrenia, memory or learning dysfunctions caused by schizophrenia, senile dementia, attention deficit/hyperactivity disorder (ADHD), central nervous system (CNS) disorder responsive to modulation of glutamate levels, major depressive episodes associated with bipolar I disorder and other associated CNS disorders.

### CROSS REFERENCE TO RELATED APPLICATIONS

This application claims priority from an Indian Patent Application IN 5702/CHE/2013 filed on Dec. 11, 2013.

### FIELD OF THE INVENTION

The present invention relates to pharmaceutical compositions comprising an atypical antipsychotic as an active agent, process of preparation thereof and method of using the same. Particularly the present invention relates to pharmaceutical compositions comprising lurasidone, process of preparation thereof and method to treat various psychotic disorders such as schizophrenia, positive and negative symptoms of schizophrenia, memory or learning dysfunctions caused by schizophrenia, senile dementia, attention deficit/hyperactivity disorder (ADHD), central nervous system (CNS) disorder responsive to modulation of glutamate levels, major depressive episodes associated with bipolar I disorder and other associated CNS disorders.

### BACKGROUND OF THE INVENTION

Psychosis refers to an abnormal condition of the mind, wherein the affected subject has a mental state that leads to “loss of contact with reality”. This usually includes false beliefs about what is taking place or who one is (delusions), seeing or hearing things that aren't existent (hallucinations) and violent outburst of emotions. Psychosis is a broad term and is often related to complex and catatonic expressions of schizophrenia and bipolar type 1 disorder.

Schizophrenia is a mental disorder characterized by a breakdown of thought processes, impaired emotional responses, loss of cognizance, judgment and motivation, behavior leading to faulty perception, inappropriate actions and feelings, withdrawal from reality and personal relationships into fantasy and delusion, and a sense of mental fragmentation. Due to many possible combinations of symptoms, there is still a debate that whether the diagnosis relates to single disorder or myriad syndromes. Treatment of this mental disorder involves use of antipsychotic medications which suppress the dopamine or serotonin activity. The primary treatment of schizophrenia is antipsychotic medications, often in combination with psychological and social supports (Van Os J, Kapur S. Schizophrenia. Lancet. 2009; 374(9690):635-45). Hospitalization may occur for severe episodes either voluntarily or (if mental health legislation allows it) involuntarily.

First line of treatment for schizophrenia involves the use of antipsychotic medications which reduce the positive symptoms of psychosis. These medications are classified as atypical as well as typical antipsychotics. Both the medications block receptors in the brain's dopamine pathways, but atypicals differ from typical antipsychotics in that they are less likely to cause extrapyramidal motor control disabilities in patients, which include unsteady Parkinson's disease-type movements, body rigidity and involuntary tremors (A roadmap to key pharmacologic principles in using antipsychotics“. Primary care companion to the Journal of clinical psychiatry 9 (6): 444-54. 2007). Atypical antipsychotics (also called second generation antipsychotics) are typically used to treat schizophrenia or bipolar disorder. The mechanism of action of these agents in the treatment of schizophrenia and bipolar depression is unknown. However, its efficacy in schizophrenia and bipolar depression could be mediated through a combination of central dopamine Type 2 (D2) and serotonin Type 2 (5HT2A) receptor antagonism.

Lurasidone hydrochloride is an atypical antipsychotic belonging to the chemical class of benzisothiazol derivatives and is approved for the treatment of schizophrenia and bipolar I disorder. Like other atypical antipsychotics, lurasidone possesses dopamine (D2) and serotonin (5HT2A) antagonism but exhibits little affinity for histamine (H1), (α1)-adrenergic, or cholinergic M1 receptors. Additionally, it is a potent (5-HT7) antagonist, which may impact depression and cognition.

The chemical name of lurasidone is (3aR,4S,7R,7aS)-2-{(1R,2R)-2-[4-(1,2-benzisothiazol-3-yl)piperazin-1-ylmethyl] cyclohexylmethyl}hexahydro-4,7-methano-2H-isoindole-1,3-dione hydrochloride. Its molecular formula is C28H36N4O2S.HCl and its molecular weight is 529.14. The chemical structure is:

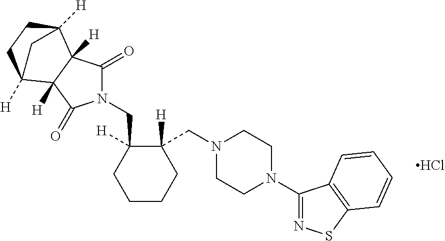

Lurasidone is currently marketed as Latuda® tablets and indicated for the treatment of patients with schizophrenia. The tablet formulation contains 20 mg, 40 mg, 60 mg, 80 mg, or 120 mg of lurasidone hydrochloride and the following ingredients: mannitol, pregelatinized starch, croscarmellose sodium, hypromellose, magnesium stearate, Opadry® and carnauba wax. Additionally, the 80 mg tablet contains yellow ferric oxide and FD&C Blue No. 2 Aluminum Lake. U.S. Pat. No. 5,532,372 patent discloses Lurasidone as an antipsychotic.

PCT WO2012/063246 & WO2012/123858 publications disclose amorphous forms of lurasidone hydrochloride. PCT WO2012/107890 & WO2013/030722 disclose various crystalline forms of lurasidone hydrochloride with percentage purity of API and specific XRD data. PCT WO2013/121440 discloses macrocrystalline lurasidone hydrochloride having particle size d90 less than about 10 microns and crystalline form of lurasidone hydrochloride with specific XPRD data. U.S. Pat. No. 7,727,553 patent discloses oral preparations with favorable disintegration characteristics wherein the compositions comprises granules having a water soluble excipient, a first disintegrant, a water soluble binder, and a second disintegrant wherein the granules are mixed with second disintegrant.

US 2009/0143404 and its family patent publication discloses an oral preparation which comprises lurasidone, a pregelatinized starch, a water soluble excipient and a water soluble polymer binder; wherein the content of lurasidone in the preparation is 20-45% w/w, and the pregelatinized starch is incorporated in an amount of 10 to 50% w/w based on the weight of the preparation. The major objective of the said patent publication was to provide an oral preparation comprising lurasidone which shows a rapid dissolution and has an equivalent dissolution profile even though contents of the active ingredient therein are varied in the wide range, with increased contents of the active ingredient which has a similar dissolution profile to that of multiple tablets with a lower content of the active ingredient per tablet and can release the active ingredient therefrom at a desired concentration. It is stated in the prosecution of the said patent that with high content of the active agent and relatively high content of pregelatinized starch taken together, said preparation would result in faster disintegration and dissolution and would tend to avoid administration of multiple tablets at one time or using a tablet having a big size which is difficult to administer.

The disclosure made in the prior art result in compositions that utilize high content of the active agent and relatively high content of pregelatinized starch in order to achieve faster disintegration and dissolution, which however unnecessarily increases the weight of the tablet and is not economically and commercially viable. Still there is a need of alternate preparations which do not require high content of starch derivatives, which show rapid dissolution and have desired bioavailability, and which avoids the need of administration of multiple tablets at one time or using a tablet having a big size that is difficult to administer. Inventors of the instant invention with considerable expense of intellectual effort and careful experimentation have developed compositions comprising lurasidone and starch derivatives which are simple and economical, and which resolve the problems of the prior art described herein above.

### SUMMARY OF THE INVENTION

An aspect of the present invention provides oral pharmaceutical compositions comprising an atypical antipsychotic as an active agent(s), starch or starch derivative(s), optionally with one or more other pharmaceutically acceptable excipient(s).

An aspect of the present invention provides oral pharmaceutical compositions comprising an lurasidone as an active agent(s), starch or starch derivative(s), optionally with one or more other pharmaceutically acceptable excipient(s).

An aspect of the present invention provides oral pharmaceutical compositions comprising lurasidone as an active agent(s) from about 0.1% w/w to about 90% w/w of the composition, starch or starch derivative(s) in an amount less than about 10% w/w of the composition, optionally with one or more other pharmaceutically acceptable excipient(s).

An aspect of the present invention provides oral pharmaceutical compositions comprising lurasidone as an active agent(s) from about 0.1% w/w to about 90% w/w of the composition, starch or starch derivative(s) in an amount more than about 50% w/w of the composition, optionally with one or more other pharmaceutically acceptable excipient(s).

An aspect of the present invention provides oral pharmaceutical compositions comprising lurasidone as an active agent(s) from about 0.1% w/w to about 90% w/w of the composition, pregelatinized starch present in an amount less than about 10% w/w of the composition, optionally with one or more other pharmaceutically acceptable excipient(s).

An aspect of the present invention provides oral pharmaceutical compositions comprising lurasidone as an active agent(s) from about 0.1% w/w to about 90% w/w of the composition, pregelatinized starch present in an amount more than about 50% w/w of the composition, optionally with one or more pharmaceutically acceptable excipient(s).

In an aspect, the present invention provides process for the preparation of stable oral pharmaceutical compositions, wherein the process comprises of the following steps:
(i) treating lurasidone with starch or starch derivative(s),
(ii) optionally adding one or more other pharmaceutically acceptable excipients, and
(iii) formulating the material of step (i) and (ii) into a suitable dosage form.

An aspect of the present invention relates to method of using such compositions indicated to treat various psychotic disorders such as schizophrenia, positive and negative symptoms of schizophrenia, memory or learning dysfunctions caused by schizophrenia, senile dementia, attention deficit/hyperactivity disorder (ADHD), central nervous system (CNS) disorder responsive to modulation of glutamate levels, major depressive episodes associated with bipolar I disorder and other associated CNS disorders.

### DETAILED DESCRIPTION OF THE INVENTION

The term “particle size” unless indicated otherwise in the specification relates to particles of lurasidone free base as well as pharmaceutically acceptable salt, amorphous or crystalline, anhydrous, esters, or isomer or derivative, hydrate, prodrug or solvates thereof. Lurasidone with specific “particle size” and distribution, or surface area would provide a fast dissolution of the active ingredient, would be easy to prepare and stable while maintaining the beneficial properties with respect to fast solubility and bioavailability. Particularly, according to the present invention, lurasidone having an average particle size less than about 200 microns, and/or surface area less that about 5 m²/gm are useful.

The term “composition” or “pharmaceutical composition” or “dosage form” as used herein synonymously include solid dosage forms such as granules, multiunit particulate systems (MUPS), pellets, spheres, tablets, capsules, mini-tablets, layered tablets, beads, particles and the like; and liquid dosage forms such as solutions, suspensions, emulsions, colloids and the like, meant for oral administration.

The term “excipient” means a pharmacologically inactive component such as a diluent, disintegrant, carrier, or the like. The excipients that are useful in preparing a pharmaceutical composition are generally safe, non-toxic and are acceptable for veterinary as well as human pharmaceutical use. Reference to an excipient includes both one and more than one such excipient.

The term “lurasidone” unless indicated otherwise in the entire specification, refers to lurasidone in its free base form, or as a pharmaceutically acceptable salt, amorphous or crystalline, anhydrous, esters, or isomer or derivative, hydrate, prodrug or solvates thereof. Preferably lurasidone is in the form of a pharmaceutically acceptable acid addition salt, more preferably, in the form of its hydrochloride salt. The phrase “substantially pure polymorphic form of lurasidone or its salt thereof”, unless otherwise specified is to be understood as a substance free of other polymorphic and/or pseudopolymorphic forms at amounts detectable with typical analytical methods such as X-ray powder diffraction and/or solid state infrared absorption, i.e. containing less than 10% of other polymorphic and/or pseudopolymorphic forms.

The present invention provides oral pharmaceutical compositions comprising an atypical antipsychotic as an active agent(s), starch or starch derivative(s), optionally with one or more other pharmaceutically acceptable excipient(s).

In an embodiment the present invention provides oral pharmaceutical compositions comprising lurasidone as an active agent(s), starch or starch derivative(s), optionally with one or more other pharmaceutically acceptable excipient(s).

In one of the embodiments the present invention provides oral pharmaceutical compositions comprising lurasidone as an active agent(s) from about 0.1% w/w to about 90% w/w of the composition, starch or starch derivative(s) in an amount less than about 10% w/w or more than about 50% w/w of the composition, optionally with one or more other pharmaceutically acceptable excipient(s).

In an embodiment, lurasidone as an active agent(s) is present in an amount less than about 50% w/w of the composition. In another embodiment, lurasidone as an active agent(s) is present in an amount between about 20% w/w and about 50% w/w of the composition.

In yet another embodiment, starch or starch derivative(s) is present in an amount of less than about 10% w/w of the composition. In one of embodiments, starch or starch derivative(s) is present in an amount of more than about 50% w/w of the composition.

In an embodiment, the starch or starch derivative(s) useful in the present invention are selected from but not limited to corn starch, maize starch, waxy corn, high amylose corn, potato, tapioca, rice, sago, pregelatinised starch, wheat or waxy sorghum starch and the like used either alone or in combination thereof. In an embodiment, the preferred starch or starch derivative is pregelatinised starch.

In another embodiment, the present invention provides pharmaceutical compositions comprising lurasidone having an average particle size of about 1 μm to about 200 μm, whereby any combinations of the lower and upper limits of these ranges are also intended to be covered by the present invention.

In an embodiment, the present invention provides process for the preparation of oral pharmaceutical compositions, wherein the process comprises of the following steps:
(i) treating lurasidone with starch or starch derivative(s),
(ii) optionally adding one or more other pharmaceutically acceptable excipients, and
(iii) formulating the material of step (i) and step (ii) into a suitable dosage form.

As used herein, unless indicated otherwise, references to total weight of the pharmaceutical composition refers to the total weight of the active agent(s) and pharmaceutically acceptable excipient(s).

“Pharmaceutically acceptable excipient(s)” are components that are added to the pharmaceutical formulation other than the active ingredient lurasidone. Excipients may be added to facilitate manufacture, enhance stability, enhance product characteristics, enhance bioavailability, enhance patient acceptability, etc. Pharmaceutically acceptable excipient(s) includes, but not limited to, one or more of diluents/fillers, binders, disintegrants, lubricants, glidants, compression aids, colors, sweeteners, preservatives, surfactants, suspending agents, dispersing agents, film formers, flavors, printing inks, and any other excipient known to the art for making pharmaceutical formulations. It will be appreciated by the person skilled in the art that a particular excipient may perform multiple roles in the pharmaceutical composition, such as for example, it can act as both a binder and filler, or as a binder and filler and disintegrant.

Binders hold the ingredients in the composition together. Exemplary binders are selected from, but not limited to, cellulose and its derivatives including, ethyl cellulose, hydroxypropyl cellulose, hydroxypropyl methylcellulose, methylcellulose and hydroxyethyl cellulose, carboxymethyl cellulose; gelatin, liquid glucose; starch and its derivatives (e.g. corn starch); hydrocolloids; sugars; polyvinyl pyrrolidone, sodium alginate, acacia, alginic acid, tragacanth, xanthan, used either alone or combinations thereof. The binder may be used in the range of about 1-10% by weight of the composition.

Diluents increase the bulk of the composition. Diluents according to the present invention are selected from, but not limited to, sugars such as lactose, sucrose, dextrose, mannose, fructose, galactose; sugar alcohols such as sorbitol, mannitol, erythritol, xylitol, lactitol; starlac, starch, modified starches, dibasic calcium phosphate, tribasic calcium phosphate, calcium carbonate, calcium sulfate, powdered cellulose, microcrystalline cellulose, silicified microcrystalline cellulose, magnesium carbonate, magnesium oxide, magnesium alumino metasilicate and the like used either alone or in combinations thereof. The diluent may be used in the range of about 20-98% by weight of the composition.

Disintegrants according to the present invention are selected from, but not limited to, cellulose and its derivatives including low-substituted hydroxypropyl cellulose, cross-linked polyvinylpyrrolidone, sodium carboxymethylcellulose, cross-linked sodium carboxymethylcellulose, microcrystalline cellulose, sodium starch glycolate, ion-exchange resins, starch and modified starches including pregelatinized starch, formalin-casein, used either alone or in combinations comprising one or more of the foregoing disintegrants. In an embodiment, the disintegrant may be used in the range of about 1-20% by weight of the composition.

Lubricants and glidants aid in the processing of powder materials. Exemplary lubricants are selected from, but not limited to, calcium stearate, glycerol behenate, magnesium stearate, mineral oil, polyethylene glycol, sodium stearyl fumarate, stearic acid, talc, vegetable oil, and zinc stearate, used either alone or in combinations comprising one or more of the foregoing lubricants. Exemplary glidants include, but not limited to, talc, silicon dioxide, cornstarch and the like used either alone or in combination thereof.

Surfactants are compounds which are capable of improving the wetting of the drug and/or enhancing the dissolution. The surfactants can be selected from hydrophilic surfactants or lipophilic surfactants or mixtures thereof. The surfactants can be anionic, nonionic, cationic, and zwitterionic surfactants. Surfactants according to the present invention are selected from, but not limited to, polyoxyethylene alkylaryl ethers such as polyoxyethylene lauryl ether, polyoxyethylene cetyl ether, polyoxyethylene stearyl ether; polyethylene glycol (PEG) fatty acid esters such as PEG monolaurate, PEG dilaurate, PEG distearate, PEG dioleate; polyoxyethylene sorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, polyoxyethylene castor oil derivates such as polyoxyl castor oil, polyoxyl hydrogenated castor oil, sodium lauryl sulphate and the like used either alone or in combination thereof.

“Suitable solvent” according to the present invention can be any solvent in which the binder is soluble or dispersible and is selected from isopropyl alcohol, ethanol, water, acetone, methylene chloride and the like or mixtures thereof.

It must be appreciated that the pharmaceutical compositions of the present invention can include all the dosage forms known to a person skilled in art, viz. formulations such as single unit dosage forms in the form of tablets, bilayer tablets, inlaid tablets, tablet in tablet, multilayered tablets, minitablets filled in capsules and the like; beads, pellets presented in a sachet, capsule or tablet capsules such as soft and hard gelatin; lozenges or sachets; granulates, microparticles, multiparticulates, powder and the like. The tablets in accordance with the present invention can be prepared by either direct compression, dry compression (slugging), or by wet granulation. The wet granulation method may comprise use of aqueous solvent such as water or an organic solvent such as ethanol, or a mixture thereof as the granulating aid.

In an embodiment, the compositions of the present invention may additionally comprise of a colorant in order to produce a desirable color. Colors known to be ‘FD&C’ certified may be used to provide coloring to the product and are within the purview of the present invention. Suitable colorants include natural colorants i.e., pigments and dyes obtained from mineral, plant, and animal sources. Examples of natural colorants include red ferric oxide, yellow ferric oxide, annattenes, alizarin, indigo, rutin, quercetin, and the like. Synthetic colorants may also be used, which is typically an FD&C or D&C dye, e.g., an approved dye selected from the so-called ‘coal-tar’ dyes, such as a nitroso dye, a nitro dye, an azo dye, an oxazine, a thiazine, a pyrazolone, a xanthene, an indigoid, an anthraquinone, an acridine, a rosaniline, a phthalein, a quinoline, or a ‘lake’ thereof, i.e. an aluminum or calcium salt thereof. Particularly preferred colorants are food colorants in the ‘GRAS’ (Generally Regarded as Safe) category.

In an embodiment, the tablet compositions of the present invention may be film coated. A film forming agent may provide smooth film-forming coating suspensions and enhance the rheological mechanical strength properties of film coating gel matrices. Film forming agents include, for example, polyvinylpyrrolidone, natural gums, starches, and cellulosic polymers. A cellulosic polymer may include a molecule comprising at least one cellulose polymer or derivative modified with small amounts of propylene glycol ether groups attached to the cellulose anhydroglucose chain affording binding properties that enhance the reinforcing film properties of film applications. Examples of cellulosic polymers include, but are not limited to, hydroxypropyl methyl cellulose (“HPMC”), carboxymethyl cellulose (“CMC”) or salts thereof, hydroxypropyl cellulose (“HPC”), methylcellulose (“MC”), hydroxyethyl cellulose (“HEC”), and the like. In addition, cellulosic polymers may be characterized as ionic or non- ionic. Ionic cellulosic polymers include, for example, sodium CMC. Non-ionic cellulosic polymers include, for example, HPMC, HPC, HEC, and MC. Varieties of commercially available cellulosic polymers exist and may include, for example, Spectracel® HPMC compositions (available from Sensient Technologies). Further, other commercially available coating materials are available marketed under the brand name Opadry® for example Opadry II Gray which contains: lactose monohydrate NF, hypromellose type 2910 USP, titanium dioxide USP, triacetin USP, and iron oxide black JPE; Opadry II Pink which contains: hypromellose type 2910 USP, titanium dioxide USP, lactose monohydrate NF, polyethylene glycol 3350 NF, triacetin USP, and FD&C Red #40; Opadry II Blue which contains: hypromellose type 2910 USP, lactose monohydrate NF, FD&C Blue #1, polyethylene glycol 3350 NF, FD&C Blue #2, titanium dioxide USP, triacetin USP, and D&C Yellow #10; Opadry II Yellow which contains: hypromellose type 2910 USP, lactose monohydrate NF, titanium dioxide USP, iron oxide yellow NF, polyethylene glycol 3350 NF, and triacetin USP; Opadry II Purple which contains: hypromellose type 2910 USP, lactose monohydrate NF, titanium dioxide USP, D&C Red #27, polyethylene glycol 3350 NF, triacetin USP, and FD&C Blue #1 and the like.

It is also desirable that the compositions are chemically stable as degradation by oxidation, hydrolysis, isomerisation, photolysis, polymerization, or any other method of degradation, either as a result of mixing with excipients or by any other method, could lead to a change in bioavailability and could also lead to toxicity. Chemical stability can be measured by a suitable, stability indicating chromatographic method for determining degradation products (see Aulton Me., Pharmaceutics—The Science of Dosage Form Design, 2.sup.nd Edition, 2002, Churchill Livingstone).

In another embodiment, the present invention provides a method of using such compositions indicated to treat various psychotic disorders such as schizophrenia, positive and negative symptoms of schizophrenia, memory or learning dysfunctions caused by schizophrenia, senile dementia, attention deficit/hyperactivity disorder (ADHD), central nervous system (CNS) disorder responsive to modulation of glutamate levels, major depressive episodes associated with bipolar I disorder and other associated CNS disorders, using the compositions of the present invention.

The compositions of the present invention can be packed into suitable containers such as bottles, blisters or pouch. Further, the packages may optionally contain a dessicant or an antioxidant or oxygen absorbant or combinations thereof.

The following examples serve to illustrate the embodiments of the present invention. However, they do not intend to limit the scope of the invention. It is obvious to those skilled in the art to find out the composition for other dosage forms and substitute the equivalent excipients as described in this specification or with the one known to the industry.

### EXAMPLE 1

| | |
|---|---|
| Ingredients | % age (w/w) |

Intra granular ingredients

| | | |
|---|---|---|
| 1. | Lurasidone hydrochloride | 25.00 |
| 2. | Sorbitol | 35.00 |
| 3. | Maize starch | 8.00 |
| 4. | Sodium starch glycollate | 4.90 |

Binder solution

| | | |
|---|---|---|
| 5. | Povidone | 7.65 |
| 6. | Purified water* | q.s. |

Extra granular Ingredients

| | | |
|---|---|---|
| 7. | Mannitol | 17.95 |
| 8. | Zinc stearate | 1.50 |

Film Coating

| | | |
|---|---|---|
| 9. | Opadry ® II yellow | q.s. for 1.5-2.5% |
| 10. | Purified water* | weight build up |

*Lost in processing
“q.s.”—quantity sufficient

### Manufacturing Process:

i). Lurasidone hydrochloride, sorbitol, maize starch and sodium starch glycollate were mixed.
ii). Binder solution was prepared by dissolving povidone in purified water.
iii). The blend of step (i) was granulated with binder solution of step (ii).
iv). The granules formed were dried and then mixed with extragranular part of mannitol.
v). The material of step (iv) was lubricated with zinc stearate.
vi). Lubricated blend was then compressed into tablets.

### Coating:

vii). Coating material was dispersed in required quantity of purified water under stirring and then stirring continued for another 45 minutes to form homogeneous dispersion.
viii). Compressed tablets of step (vi) were coated with coating dispersion of step (vii) and continued till the target weight build up was achieved.

### EXAMPLE 2

| | |
|---|---|
| Ingredients | % age (w/w) |

Intra granular ingredients

| | | |
|---|---|---|
| 1. | Lurasidone hydrochloride | 17.00 |
| 2. | Sucrose | 43.00 |
| 3. | Potato starch | 8.50 |
| 4. | Croscarmellose sodium | 3.50 |

Binder dispersion

| | | |
|---|---|---|
| 5. | Hydroxypropyl methycellulose | 6.50 |
| 6. | Isopropyl alcohol* | q.s. |

Extra granular Ingredients

| | | |
|---|---|---|
| 7. | Mannitol | 20.00 |
| 8. | Glyceryl behenate | 1.50 |

*Lost in processing.

### Manufacturing Process:

i). Lurasidone HCl, sucrose, potato starch and croscarmellose sodium were mixed.
ii). Binder solution was prepared by dissolving hydroxypropyl methycellulose in isopropyl alcohol.
iii). The blend of step (i) was granulated with binder solution of step (ii).
iv). The granules formed were dried and then mixed with extragranular part of mannitol.
v). The material of step (iv) was lubricated with glyceryl behenate.
vi). Lubricated blend was then compressed into tablets.

### EXAMPLE 3

| | |
|---|---|
| Ingredients | % age (w/w) |

Intra granular ingredients

| | | |
|---|---|---|
| 1. | Lurasidone hydrochloride | 16.50 |
| 2. | Fructose | 41.00 |
| 3. | Pregelatinised starch | 12.50 |
| 4. | Crospovidone | 3.50 |
| 5. | Sodium alginate | 9.00 |

Extra granular Ingredients

| | | |
|---|---|---|
| 6. | Xylitol | 16.00 |
| 7. | Calcium stearate | 1.50 |

### Manufacturing Process:

i) Lurasidone hydrochloride, fructose, pregelatinised starch, crospovidone and sodium alginate were sifted together.
ii) The blend of step (i) was slugged/compacted and milled.
iii) The granules obtained in step (ii) were blended with xylitol.
iv) The material of step (iii) was lubricated with calcium stearate.
v) The lubricated material of step (iv) was compressed to tablets or filled into capsules.

### EXAMPLE 4

| | |
|---|---|
| Ingredients | % age (w/w) |

Intra granular ingredients

| | | |
|---|---|---|
| 1. | Lurasidone hydrochloride | 17.00 |
| 2. | Lactose | 9.00 |
| 3. | Starch | 55.00 |
| 4. | Sodium starch glycollate | 3.50 |
| 5. | Tragacanth | 9.00 |

Extra granular Ingredients

| | | |
|---|---|---|
| 6. | Xylitol | 5.00 |
| 7. | Stearic acid | 1.50 |
| 8. | Opadry ® II purple | q.s. for 1.5-2.5% |
| 9. | Purified water* | weight build up |

*Lost in processing;
“q.s.”—quantity sufficient

### Manufacturing Process:

i) Lurasidone hydrochloride, lactose, starch, sodium starch glycollate and tragacanth were sifted together.
ii) The blend of step (i) was slugged/compacted and milled.
iii) The granules obtained in step (ii) were blended with xylitol.
iv) The material of step (iii) was lubricated with stearic acid.
v) The lubricated material of step (iv) was compressed into tablets or filled into capsules.

### Coating:

vi) Coating material was dispersed in required quantity of purified water under stirring and then stirring continued for another 45 minutes to form homogeneous dispersion.
vii) Compressed tablets of step (vi) were coated with coating dispersion and continued till the target weight build up was achieved.

### EXAMPLE 5

| | |
|---|---|
| Ingredients | % age (w/w) |

Intra granular ingredients

| | | |
|---|---|---|
| 1 | Sorbitol | 44.20 |
| 2 | Maize starch | 8.34 |
| 3 | Sodium starch glycollate | 4.90 |

Binder dispersion

| | | |
|---|---|---|
| 4 | Hydroxypropyl methylellulose | 5.96 |
| 5 | Lurasidone hydrochloride | 17.00 |
| 6 | Purified water* | q.s. |

Extra granular Ingredients

| | | |
|---|---|---|
| 7 | Erythritol | 18.38 |
| 8 | Zinc stearate | 1.22 |

Film Coating

| | | |
|---|---|---|
| 9 | Hypromellose | q.s. for 1.5-2.5% weight |
| 10 | Polyethylene glycol | build up |
| 11 | Titanium dioxide | |
| 12 | Purified water* | |

*Lost in processing.

### Manufacturing Process:

i). Sorbitol, maize starch and sodium starch glycollate were sifted together.
ii). Binder solution was prepared by dissolving hydroxypropyl methylcellulose in purified water.
iii). Lurasidone hydrochloride was added to the binder solution under stirring condition to make a homogeneous dispersion.
iv). The blend of step (i) was granulated with material of step (iii).
v). The granules formed were dried and then mixed with extragranular part of erythritol.
vi). The material of step (v) was lubricated with zinc stearate.
vii). Lubricated blend was then compressed into tablets.

### Coating:

viii). Coating material was dispersed in required quantity of purified water under stirring and then stirring continued for another 45 minutes to form homogeneous dispersion.
ix). Compressed tablets of step (vii) were coated with coating dispersion and continued till the target weight build up was achieved.

### EXAMPLE 6

| | |
|---|---|
| Ingredients | % age (w/w) |

Intra granular ingredients

| | | |
|---|---|---|
| 1 | Mannitol | 36.70 |
| 2 | Pregelatinized starch | 8.34 |
| 3 | Crospovidone | 4.90 |

Binder dispersion

| | | |
|---|---|---|
| 4 | Hydroxypropyl cellulose | 7.65 |
| 5 | Lurasidone hydrochloride | 24.50 |
| 6 | Purified water* | q.s. |

Extra granular Ingredients

| | | |
|---|---|---|
| 7 | Mannitol | 18.38 |
| 8 | Magnesium stearate | 1.22 |

Film Coating

| | | |
|---|---|---|
| 9 | Hypromellose | q.s. for 1.5-3.0% weight |
| 10 | Polyethylene glycol | build up |
| 11 | Titanium dioxide | |
| 12 | Purified water* | |

*Lost in processing

### Manufacturing Process:

i). Mannitol, pregelatinised starch and crospovidone were sifted together.
ii). Binder solution was prepared by dissolving hydroxypropyl cellulose in purified water.
iii). Lurasidone hydrochloride was added to the binder solution under stirring condition to make a homogeneous dispersion.
iv). The blend of step (i) was granulated with material of step (iii).
v). The granules formed were dried and then mixed with extragranular part of mannitol.
vi). The material of step (v) was lubricated with magnesium stearate.
vii). Lubricated blend was then compressed into tablets.
viii). Coating material was dispersed in required quantity of purified water under stirring and then stirring continued for another 45 minutes to form homogeneous dispersion.
ix). Compressed tablets of step (vii) were coated with coating dispersion and continued till the target weight build up was achieved.

We claim:

1. An oral pharmaceutical composition comprising an atypical antipsychotic as an active agent, starch or a starch derivative, optionally with at least one or more other pharmaceutically acceptable excipient.

2. The composition according to claim 1, wherein the said active agent is lurasidone.

3. The composition according to claim 1, comprising lurasidone as an active agent from about 0.1% w/w to about 90% w/w of the composition, starch or starch derivative in an amount less than about 10% w/w or more than about 50% w/w of the composition, optionally with one or more other pharmaceutically acceptable excipient.

4. The composition according to claim 2, wherein lurasidone is present in an amount of less than about 50% w/w of the composition.

5. The composition according to claim 2, wherein lurasidone as an active agent is present in an amount between about 20% w/w and about 50% w/w of the composition.

6. The composition according to claim 1, wherein starch or starch derivative(s) is present in an amount less than about 10% w/w of the composition.

7. The composition according to claim 1, wherein starch or starch derivative(s) is present in an amount more than about 50% w/w of the composition.

8. The composition according to claim 1, wherein the starch or starch derivative are selected from a group comprising corn starch, maize starch, waxy corn, high amylose corn, potato, tapioca, rice, sago, pregelatinised starch, wheat or waxy sorghum starch used either alone or in combination thereof.

9. The composition according to claim 1, wherein the pharmaceutically acceptable excipient is selected from a group comprising diluents/fillers, binders, disintegrants, lubricants, glidants, compression aids, colors, sweeteners, preservatives, surfactants, suspending agents, dispersing agents, film formers, flavors, printing inks and combinations thereof.

10. A process for the preparation of oral pharmaceutical composition of claim 1 comprising:
i). treating lurasidone with starch or starch derivative,
ii). optionally adding one or more other pharmaceutically acceptable excipients, and
iii). formulating the material of either step (i) and step (ii) into a suitable dosage form.

11. A method of using the composition of claim 1 for the treatment of various psychotic disorders such as schizophrenia, positive and negative symptoms of schizophrenia, memory or learning dysfunctions caused by schizophrenia, senile dementia, attention deficit/hyperactivity disorder (ADHD), central nervous system (CNS) disorder responsive to modulation of glutamate levels, major depressive episodes associated with bipolar I disorder and other associated CNS disorders.

12. A method of treating one or more psychotic disorders including schizophrenia, positive and negative symptoms of schizophrenia, memory or learning dysfunctions caused by schizophrenia, senile dementia, attention deficit/hyperactivity disorder (ADHD), central nervous system (CNS) disorder responsive to modulation of glutamate levels, major depressive episodes associated with bipolar I disorder and other associated CNS disorders, by administering a composition according to claim 1 to a subject in need thereof.
